(19) United States
(12) Patent Application Publication
(10) Pub. No.: US 2009/0234360 A1
(43) Pub. Date: Sep. 17, 2009

(54) LASER ASSISTED TOTAL JOINT ARTHROPLASTY

(76) Inventor: Vladimir Alexander, Largo, FL (US)

Correspondence Address: SMITH HOPEN, PA, 180 PINE AVENUE NORTH, OLDSMAR, FL, 34677, US

(21) Appl. No.: 11/955,378

(22) Filed: Dec. 12, 2007

Related U.S. Application Data
(60) Provisional application No. 60/869,601, filed on Dec. 12, 2006.

(51) Int. Cl. A61B 17/90

(52) U.S. Cl. 606/88; 606/89; 606/87

(57) ABSTRACT

The present invention provides an improved method of performing bone resection, including during arthroplasty procedures, wherein a laser guide is utilized to align cutting blocks and/or measuring devices. The present invention also provides laser guidance platforms for use in the methods of the invention.

### REFERENCE TO RELATED APPLICATIONS AND INCORPORATION BY REFERENCE

This application claims priority to U.S. Provisional Patent Application No. 60/869,601, filed Dec. 12, 2006.

The above referenced application and each document cited in this text (“herein cited documents”) and each document cited or referenced in each of the herein cited documents, including any product specifications or instruction manuals, are hereby incorporated herein by reference. Furthermore, each of the applications and patents and documents referenced in the present application, and each document cited or referenced in each of these applications and patents, including during any prosecution (“application cited documents”), and each document cited or referenced in each of the application cited documents, are additionally hereby incorporated herein by reference.

### FIELD OF THE INVENTION

The present invention generally concerns the field of joint replacement surgery, and more particularly, resides in improved apparatus and methods of performing bone resection to better assist a surgeon in preparing a bone, for example, to receive an implant. A particular embodiment of the invention being described, without limitation, provides improved methods of performing bone resection in the context of knee arthroplasty. More particularly, with respect to knee arthroplasty, the invention relates to methods and devices used to properly locate and guide instruments that resect bone in order to achieve a proper cut; and facilitate the proper location and installation of artificial femoral and tibial prosthetic components.

### BACKGROUND OF THE INVENTION

The goal of arthroplasty is to restore the function of a stiffened, diseased or damaged synovial joint and relieve pain. Synovial joints are those which contain a cushion of synovial fluid between the bones creating the joint, which includes, but is not limited to, knees, hips, ankles, shoulders and elbows. As a surgical procedure, it is usually performed following a lack of success with non-surgical treatment options.

Types of arthroplastic surgery include joint resection and interpositional reconstruction. Joint resection involves removing a portion of the bone from a stiffened joint, and increasing the space between the bone and the socket to improve the range of motion. Scar tissue eventually fills the gap, narrowing the joint space again. Pain is relieved and motion is restored, but the joint is less stable.

Interpositional reconstruction is surgery to reshape the joint and add a prosthetic disk between the two bones forming the joint. The prosthesis can be made of plastic, metal, ceramic material, or formed from such body tissue as skin, muscle, or fascia. When interpositional reconstruction fails, total joint replacement may be necessary. Joint replacement is also called total joint arthroplasty.

In recent years, joint replacement has become the operation of choice for most chronic knee and hip problems, particularly because of advances in the type and quality of prostheses (artificial joints). Elbow, shoulder, ankle, and finger joints are more likely to be treated with joint resection or interpositional reconstruction, although all may be candidates for total joint arthroplasty as well.

Arthroplasty is often performed on people suffering from severe pain and disabling joint stiffness. Osteoarthritis, a degenerative joint disease, is the most common condition causing joint destruction with pain and impaired movement. Other causes include rheumatoid arthritis, hemophilia, synovitis, and rare bone diseases, which are all known to destroy cartilage. Arthoplasty is additionally performed in response to injuries which impact the function of the joint.

Total joint arthoplasty is traditionally an extremely invasive procedure, although minimally-invasive techniques have recently been developed. Total joint arthroplasty necessarily requires the removal of at least a portion of the bone material that comprises the joint to be replaced, which removal must be carefully performed to allow the fitting of the replacement joint.

For example, total knee arthroplasty involves the replacement of portions of the patella, femur and tibia with artificial components. In particular, a proximal portion of the tibia and a distal portion of the femur are cut away (resected) and replaced with artificial components.

There are several types of knee prostheses known in the art. One type is sometimes referred to as a “resurfacing type”. In these prostheses, the articular surface of the distal femur and proximal tibia are “resurfaced” with respective metal and plastic condylar-type articular bearing components.

The femoral component is typically a metallic alloy construction (often cobalt-chrome alloy or 6A14V titanium alloy) and provides medial and lateral condylar bearing surfaces of multi-radius design of similar shape and geometry as the natural distal femur or femoral-side of the knee joint.

One important aspect of these procedures is the correct resection of the distal femur and proximal tibia. These resections must provide planes which are correctly angled in order to properly accept the prosthetic components. FIGS. 15A and B show a femur and tibia, respectively, that have been resected and fit with prosthetic components. The complexity of the resection of each bone is visible in this Figure. In particular, the resection planes must be correctly located relative to three parameters: proximal-distal location, varus-valgus angle and flexion-extension angle.

Some devices of the prior art (including U.S. Pat. No. 5,916,219) provide an apparatus and method for tibial alignment which allows the independent establishment of two separate geometric planes to be used as a reference for the cutting of the tibial plateau during total knee arthroplasty.

In one system of the prior art, two separate frame assemblies with telescoping rods are attached to the tibia with a fixed relative angle between them, thereby allowing alignment with the mechanical axis of the bone. A cutting block is mounted on one of the assembly frames and is positioned against the tibia. Stabilizing pins are then placed in the cutting block, allowing the proper tibial plateau resection plane to be created.

One system of measuring devices and cutting guides is shown in FIG. 13 (femur) and FIG. 14 (tibia).

However, many devices available in the prior art have a few disadvantages. Typically, the alignment apparatus must be removed prior to performing resection. Many devices ratchet to discrete locations, preventing a smooth alignment. Further, some devices can only be used to resect the tibia and cannot be used for femoral resection.

Recently, various computerized systems have been introduced to aid the practitioner during different surgical procedures. Such systems are readily commercially available. Such systems typically include multiple video cameras which are deployed above and around the surgical site; and a plurality of dynamic reference frame (DRF) devices, also known as trackers, which are attached to body parts and surgical instruments.

The trackers are generally LED devices which are visible to the cameras. Using software designed for a particular surgical procedure, a computer receiving input from the cameras guides the placement of surgical instruments.

However, the prior art instruments used for determining the correct planes for tibial and femoral resection in total knee arthroplasty are not well suited for use with computerized systems. The known tools utilize either intra-medullary alignment or extra-medullary alignment techniques and movement in three degrees of freedom is difficult or impossible. Moreover, in order to be useful with computer aided navigation systems, trackers must be attached to the tools. Existing tools do not permit or readily facilitate the attachment of trackers.

Although computer aided navigation systems are superior to unaided visual navigation by the practitioner, computers are not infalliable. A computer can crash or fail in such a way that it may take significant time to repair, which is unacceptable during a surgical procedure.

Therefore, there is a need in the art for an alternative system of conducting bone resection which is not reliant on computer-assisted technology, which is easy to use and accurate when used with a large population of patients, and which reduces the number of instruments associated with a typical joint surgery. Although described above with relation to total knee replacements, any joint replacement surgery faces the same types of challenges which are not currently addressed by prior art systems and which are alleviated by the system of the present invention.

### SUMMARY OF THE INVENTION

The present invention provides a method of performing a laser assisted total joint arthroplasty comprising the use of a laser guidance system to determine the location and/or angle and/or depth of those cuts in the bone required to fit a replacement joint to a subject in need thereof.

In one aspect of the present invention, the laser guidance system comprises one or more lasers affixed to a cutting block and/or a measuring device via an attachment means. In another embodiment, the lasers can be aligned to illuminate the appropriate plane necessary for resection, including, for example, the sagittal or coronal planes.

In another aspect of the present invention, the laser guidance system of the present invention can be easily adapted for use in joint replacements on either the left or right side of the patient's body.

In yet another aspect of the present invention, the laser guidance system additionally comprises an attaching means for attaching the laser guidance system to the bone. In a further aspect of the invention, the attaching means is engaged following alignment of the laser to lock the cutting block and/or measuring device into the appropriate position. In yet a further aspect of the invention, the laser guidance system does not have to be removed prior to resection of the bone.

In a further aspect of the invention, the laser guidance system can be attached to existing cutting blocks and/or measuring devices via the attachment means. Alternatively, the laser guidance system can be provided as part of a kit comprising cutting blocks and/or measuring devices specifically adapted for use with the laser guidance system.

In one aspect of the invention, the methods and laser guidance system of the present invention can be utilized in any type of arthroplasty, including total knee arthroplasty, total hip arthroplasty, total ankle arthroplasty, and total shoulder arthroplasty, etc.

The present invention further provides for a laser guidance system comprising one or more lasers and an attaching means, wherein the attaching means can be secured to a cutting block and/or measuring device. In a further aspect of the invention, the laser guidance system additionally comprises an additional attaching means whereby the laser guidance system can be secured to the bone.

In one aspect of the invention, the laser guidance system can be disposable, or designed and/or manufactured specifically for a single use. In another aspect of the invention, the laser guidance system can be designed and/or manufactured to be used repeatedly.

In yet another aspect of the invention, there is provided an apparatus for guiding the resection of a bone during arthroplasty, comprising: a) anchoring means for anchoring the apparatus to the bone; b) a resection guide coupled to said anchoring means; and c) means for locating the resection guide relative to the anchoring means, said means comprising a laser guide.

In a further aspect of the invention, there is provided an apparatus for guiding the resection of a bone during arthroplasty, comprising: a) a laser guide and b) attaching means for removably attaching said laser guide to a resection guide, wherein the resection guide comprises means for attaching the resection guide to bone.

Additional features and benefits of the present invention are described, and will be apparent from, the accompanying drawings and the detailed description below. It is to be understood that both the foregoing general description and the following detailed description are exemplary and explanatory only and are not restrictive of the invention as claimed. The accompanying drawings, which are incorporated in and constitute a part of the specification, illustrate an embodiment of the invention and together with the general description, serve to explain the principles of the invention.

### DESCRIPTION OF THE DRAWINGS

The present invention will now be hereinafter described in more detail by way of example only, including by way of the attached figures in which:

FIG. 1 is a schematic depicting femoral and tibial resection guides having a laser guidance system in accordance with the present invention. The alignment of the lasers in the coronal and/or sagittal planes is depicted.

FIG. 2A and FIG. 2B are photographs of a femoral resection guide having a laser guidance system in accordance with the present invention. FIG. 2B is a rendition of FIG. 2A having identifying numbers provided therein to label the components.

FIG. 3A and FIG. 3B are photographs of a femoral resection guide having a laser guidance system in accordance with the present invention. FIG. 3B is a rendition of FIG. 3A having identifying numbers provided therein to label the components.

FIG. 4A and FIG. 4B are photographs of a femoral resection guide having a laser guidance system in accordance with the present invention. FIG. 4B is a rendition of FIG. 4A having identifying numbers provided therein to label the components.

FIG. 5A and FIG. 5B are photographs of a tibial resection guide having a laser guidance system in accordance with the present invention. FIG. 5B is a rendition of FIG. 5A having identifying numbers provided therein to label the components.

FIG. 6A and FIG. 6B are photographs of a tibial resection guide having a laser guidance system in accordance with the present invention. FIG. 6B is a rendition of FIG. 6A having identifying numbers provided therein to label the components.

FIG. 7A and FIG. 7B are photographs of a tibial resection guide having a laser guidance system in accordance with the present invention. FIG. 7B is a rendition of FIG. 7A having identifying numbers provided therein to label the components.

FIG. 8A and FIG. 8B are photographs of a tibial resection guide having a laser guidance system in accordance with the present invention. FIG. 8B is a rendition of FIG. 8A having identifying numbers provided therein to label the components.

FIG. 9A and FIG. 9B are photographs of a tibial resection guide having a laser guidance system in accordance with the present invention. FIG. 9B is a rendition of FIG. 9A having identifying numbers provided therein to label the components.

FIG. 10A and FIG. 10B are photographs of a tibial resection guide having a laser guidance system in accordance with the present invention. FIG. 10B is a rendition of FIG. 10A having identifying numbers provided therein to label the components.

FIG. 11A is a schematic depicting a tibial resection guide having attached thereto a laser guidance system in accordance with the present invention.

FIG. 11B is a schematic depicting a femoral resection guide having attached thereto a laser guidance system in accordance with the present invention.

FIG. 12A is a schematic depicting a tibial resection guide having attached thereto a laser guidance system in accordance with the present invention to demonstrate the placement of the laser in relation to the anatomy of the tibia.

FIG. 12B is a schematic depicting a femoral resection guide having attached thereto a laser guidance system in accordance with the present invention to demonstrate the placement of the laser in relation to the anatomy of the femur.

FIG. 13 is a schematic depicting standard femoral resection guides in practice.

FIG. 14 is a schematic depicting standard tibial resection guides in practice.

FIG. 15A is a schematic depicting a resected femur having attached thereto an artificial femoral component. The figure exemplifies the manner in which a femur is cut to fit the femoral component being used in the arthoplasty.

FIG. 15B is a schematic depicting a resected tibia having attached thereto an artificial tibular component. The figure exemplifies the manner in which a tibia is cut to fit the tibular component being used in the arthoplasty.

### DETAILED DESCRIPTION OF THE INVENTION

The present invention provides an improved surgical method of performing total joint arthroplasties and devices utilized therein. The present invention further provides an improved method of performing partial joint arthroplasties and devices utilized therein.

The present invention may be utilized with any type of arthroplasty, including but not limited to knee arthroplasty, hip arthroplasty, ankle arthroplasty, and shoulder arthroplasty. Furthermore, the present invention is additionally applicable to any type of surgical procedure wherein the alignment of components or instruments is necessary or desired, or wherein the precise location of incisions or cuts is necessary or desired.

Although the present invention is broadly applicable as described above, the claimed laser-guidance system will be further described herein by way of a non-limiting depiction of a knee arthroplasty and the instrumentation utilized therein. Such description is not intended to limit the present invention in any form.

As used herein, when referring to bones or other body parts, the term “proximal” means closest to the heart and the term “distal” means more distant from the heart. When referring to tools and instruments, the term “proximal” means closest to the practitioner and the term “distal” means distant from the practitioner.

As used herein, when referring to planes or sections of the body, the term “sagittal” has the definition ascribed to it in the art, namely a plane through the body separating the body into a left and right half, and the term “coronal” has the definition ascribed to it in the art, namely a plane that separates the anterior and posterior portions of the body.

The present invention can be used with any type of cutting blocks or measuring devices designed for use in bone resection, preferably those designed for use in arthroplasty. For example, cutting blocks and measuring devices from Zimmer, DePuy, Stryker Howmedica Osteonics Corp., MedIdea, LLC, U.S. Medical Products, Inc., Biomet, Sigma, Smith & Nephew, Ortho Development, Encore, Tornier, Exactech, etc., may be used with the system and instruments of the present invention.

In one embodiment of the present invention, a surgical technique is provided whereby a practitioner may utilize the laser guidance system of the present invention in combination with commercially available bone resection blocks (or “cutting blocks”) whereby the laser guidance system allows the practitioner to appropriately align the cutting block and/or measuring device by relying on the planar beam provided by the laser guidance system. In another embodiment of the present invention, a surgical technique is provided whereby a practitioner may utilize the laser guidance system of the present invention in combination with commercially available measuring devices whereby the laser guidance system allows the practitioner to appropriately align the measuring device by relying on the planar beam provided by the laser guidance system.

In another embodiment of the present invention, the laser guidance system enables the practitioner to determine the location and/or angle and/or depth of those cuts in the bone required to fit the replacement joint to a subject in need thereof.

It is within the scope of the present invention that the laser guidance system comprises one or more laser diodes affixed to a cutting block and/or measuring device via an attachment means. In one embodiment, the laser diodes may be adjustable to enable the practitioner to change the angle of the laser beam, including a planar beam, which is emitted from the diode.

In a further embodiment, one or more prisms or similar devices can be used to split and/or direct the one or more laser beams or to provide the resulting planar beam(s).

In one embodiment, the practitioner may properly adjust the position of the laser diode to alter the angle of the laser beam, including a planar beam, which beam may then be utilized by the practitioner as a reference or guide for correctly aligning the cutting block and/or measuring device over the bone. In one embodiment, the cutting block and/or measuring device is already attached to the laser guidance system and is placed over the bone to be cut and the practitioner then engages in aligning the one or more laser beams along the desired plan such that the cutting block and/or measuring device is appropriately aligned in relation to the bone, or the practitioner may move the entire cutting block and/or measuring device and laser guidance system assembly in relation to the bone until the one or more laser beams are properly aligned.

In yet another embodiment, the laser guidance system can be removably and operably engaged with the cutting block and/or measuring device, such that the laser guidance system can be positioned properly in relation to the bone, such that the cutting block and/or measuring device is then removably attached to the laser guidance system such that the cutting block and/or measuring device is in the necessary or desired alignment with the bone. In one configuration, the laser guidance system comprises a locking mechanism and/or attaching means such that once the laser guidance system is operably engaged and/or attached with the cutting block and/or measuring device, the two or more components can be locked in place to avoid accidental movement or slippage. In a further embodiment, the laser guidance system comprises an attaching means which removably attaches to the bone to allow the laser guidance system to be attached to the bone in the proper alignment, after which the cutting block and/or measuring device can then be operably engaged with the laser guidance system. In yet a further embodiment, the laser guidance system can be attached via intramedullary or extramedullary alignment options, as are known to one of skill in the art.

In yet another aspect of the invention, the attaching means can comprise any system known in the art to be useful for such purpose, including by not limited to pins, screws, clamps, snap-fit, magnets and similar devices.

In a further embodiment, the interface between the laser guidance system and the cutting block and/or measuring device can be adjusted to provide a range of angles between the laser guidance system and the cutting block and/or measuring device. For example, if the subject bone to be cut has a deformity or a variation in shape, it may be necessary to adjust the angle between the laser guidance system and the cutting block and/or measuring device in order to accommodate the bone, to create the proper alignment, or to compensate for variations in the bone.

In another embodiment, the cutting block and/or measuring device comprises an attaching means which removably attaches to the bone to allow the cutting block and/or measuring device to be attached to the bone, preferably following proper alignment utilizing the operably engaged laser guidance system. In yet a further embodiment, the cutting block and/or measuring block, either individually or in combination with an operably engaged laser guidance system can be attached via intramedullary or extramedullary alignment options, as are known to one of skill in the art.

It is within the scope of the present invention that the at least one laser diode and/or the angle between the laser guidance system and the cutting block and/or measuring device can be adjusted to provide laser beams or planar beams in the appropriate plane for use as a guide or reference when determining the alignment of the cutting block and/or measuring device and/or the laser guidance system in relation to the bone. In one embodiment, the laser diodes and/or the angle between the laser guidance system and the cutting block and/or measuring device are adjusted such that the laser beams or planar beams are in the sagittal or coronal planes.

In another embodiment, the at least one laser diodes provide laser beams or planar beams in more than one direction, for example in both the sagittal and coronal planes, simultaneously.

In a further embodiment, one or more prisms or similar devices can be used to split and/or direct the one or more laser beams to provide the resulting planar beam(s).

In a further embodiment of the present invention, the laser guidance system can be reversed for use on the left or the right side of a patient. In one embodiment, the means of attaching the laser guidance system to the cutting block allows for the reversal of the laser guidance system. In one embodiment, the laser guidance system can include a separate, additional attaching means having a reverse configuration. In another embodiment, the system of the present invention can comprise multiple laser guidance systems which comprise mirror image configurations such that one is configured for use on the left side of the body, and one is configured for use on the right side of the body.

In a further embodiment, a single laser guidance system can comprise multiple laser diodes for use on either the right or left side of the body such that the laser diodes can be utilized either simultaneously or independently, thereby allowing the practitioner to select the appropriate diodes for use in the a given procedure. In one embodiment, the laser diodes for the left side of the body may be utilized, providing the appropriate laser beams or planar beams for alignment of cutting blocks and/or measuring devices and/or resection guides and/or replacement joints on the left side of the body. In another embodiment, the laser diodes for the right side of the body may be utilized, providing the appropriate laser beams or planar beams for alignment of cutting blocks and/or measuring devices and/or resection guides and/or replacement joints on the right side of the body.

In another embodiment, a single laser guidance system can comprise one or more laser diodes in a laser housing, wherein the laser housing can be moveable. That is, the laser housing can have a first position that provides a laser plane in a first direction, and the laser housing can be rotated or otherwise moved into a second position to provide a laser plane in a second position. For example, the laser housing can be contained on a moveable arm attached to the body of a laser guide apparatus, whereby the moveable arm can rotate, pivot and/or slide in relation to the body of the apparatus, such that the direction of the laser can be altered by adjusting the position of the moveable arm. In another embodiment, a single apparatus can contain two or more lasers positioned in one or more moveable arm and/or within the body of the apparatus, such that multiple laser beams can be adjusted in relation to each other, in relation to the resection guides, or in relation to the anatomy.

The present invention therefore encompasses an apparatus for guiding the resection of a bone during arthroplasty, comprising: a) attaching means for anchoring the apparatus to the bone; b) a resection guide or cutting guide operably engaged to said attaching means; and c) means for locating the resection guide relative to the attaching means, said means comprising a laser guide.

In yet another aspect of the invention, there is provided an apparatus for guiding the resection of a bone during arthroplasty, comprising: a) a laser guide and b) attaching means for removably attaching said laser guide to a resection guide, wherein the resection guide comprises means for attaching the resection guide to bone.

In one embodiment of the present invention, the laser system of the present invention can utilize the technology currently known in the art. For example, the laser system of the present invention can be made in accordance with any of U.S. Pat. Nos. 7,134,211, 7,134,212, 7,116,697, 7,100,293, 7,073,268, 7,059,057, 7,055,252, 7,031,367, 7,027,480, 7,013,571, 6,987,789, 6,986,209, 6,964,106, 6,944,201, 6,941,665, 6,931,737, 6,915,583, 6,914,930, 6,909,551, 6,891,878, 6,880,256, or 6,856,640. Furthermore, those of skill in the art will be able to select and apply appropriate laser systems to the practice of the present invention. For example, it may be desirous to utilize a laser system which enables a user to operate perform resections through a graphical user interface communicatively coupled with a non-contact measurement and alignment device, as described in U.S. Pat. No. 7,073,268. In such a use, the laser system would include a graphical user interface which correlates user engageable selectors with a logically related menu of tool (cutting device) setting options displayed on a display screen in a high quality and easily readable format. The non-contact measurement and alignment device uses one or more lasers to determine tool settings and establish proper alignment based on user needs.

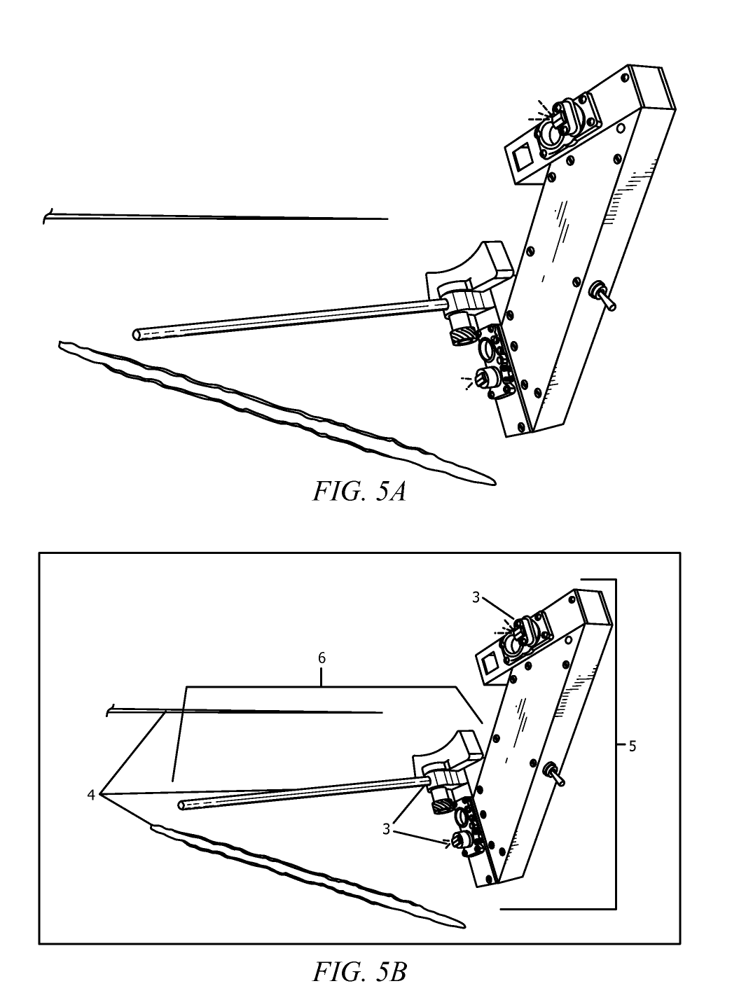

In general, the laser system of the present invention can comprise, for example, a housing, a pendulum pivotably connected to the housing, at least a first laser diode disposed on the pendulum for emitting at least a first laser beam along at least a first path, and a lens disposed on the pendulum in the at least first path for converting the at least first laser beam into a first planar beam, the at least first planar beam forming a line on the reference surface. In accordance with the present invention, the laser system can comprise more than one laser diode to provide more than one planar beams. In one embodiment, the two or more laser diodes can be contained with the same house or may be located in separate housings located some distance apart within the laser guidance system, as is shown in FIG. 5-10.

In one embodiment of the present invention, the alignment of the laser diodes can be altered, such that the resulting planar beams can be adjusted as necessary based on criteria known to those of skill in the art, for example, such criteria can include the bone to be resected, the joint being replaced, the type of replacement joint being used, the size of the patient, the size of the replacement joint, and any alterations or deformities in the bone to be resected.

In another embodiment of the present invention, the laser diodes are aligned in the sagittal or coronal planes. In another embodiment, the laser diodes provide laser beams or planar beams in more than one direction, for example in both the sagittal and coronal planes, simultaneously. In a further embodiment, one or more prisms or similar devices can be used to split and/or direct the one or more laser beams or to provide the resulting planar beam(s).

The laser beams and planar beams provided by the methods and apparatus of the present invention are also useful in determining angles incident to the surgical procedure being performed, such as the valgus angle.

The present invention also allows for the incorporation of a power supply for the one or more laser diodes to be incorporated into the apparatus or to be connected to the apparatus. For instance, the apparatus can contain batteries or another power supply connected to the one or more laser diodes. Those of skill in the art would readily appreciate the types of power supplies that are suitable for the present invention and which can be adapted for use in such an apparatus.

In another embodiment of the present invention, the laser guidance system can be disposable, or designed and/or manufactured specifically for a single use. In another aspect of the invention, the laser guidance system can be designed and/or manufactured to be used repeatedly.

It is one aspect of the present invention that the materials used in the manufacture of an apparatus as described herein will be chosen in part based upon whether the apparatus is designed for a single-use or for repeated uses. In the first instance, the apparatus can be made from plastics, polymers, resins, and the like. In the second instance, the apparatus can be made from metals, composites, alloys and the like. Alternatively, some combination of these materials can be utilized in the apparatus irrespective of the length of time the apparatus will be in use. Those of skill in the art will readily recognize alternative materials that can be used in apparatus provided for herein.

In one embodiment of the present invention, the surgical methods in accordance with the herein description and/or figures are useful in total joint arthoplasty, bone resurfacing, or other surgical techniques which require the altering the shape and/or size of bone. In a further embodiment, the total joint arthroplasty can be total knee arthroplasty, total hip arthroplasty, total ankle arthroplasty, and total shoulder arthroplasty.

In another embodiment of the present invention, a laser guidance system in accordance with the herein description and/or figures is useful in total joint arthoplasty, bone resurfacing, or other surgical techniques which require the altering the shape and/or size of bone. In a further embodiment, the total joint arthroplasty can be total knee arthroplasty, total hip arthroplasty, total ankle arthroplasty, and total shoulder arthroplasty.

In a further embodiment of the present invention, an apparatus in accordance with the herein description and/or figures is useful in total joint arthoplasty, bone resurfacing, or other surgical techniques which require the altering the shape and/or size of bone. In a further embodiment, the total joint arthroplasty can be total knee arthroplasty, total hip arthroplasty, total ankle arthroplasty, and total shoulder arthroplasty.

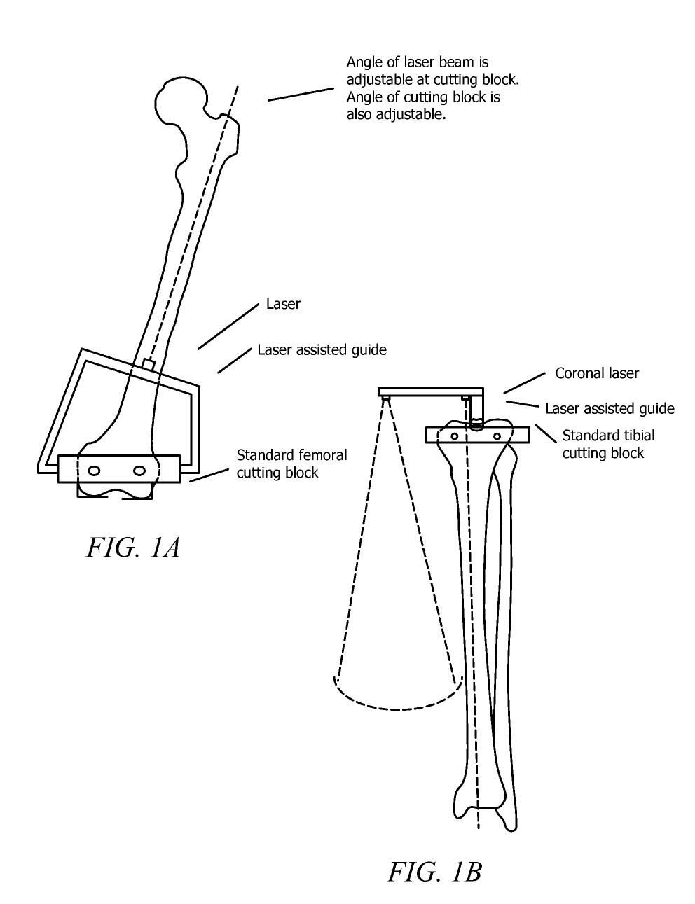

In one embodiment of the present invention, the surgical methods and/or laser guidance system and/or apparatus are useful in knee arthoplasty. In a further embodiment, the surgical methods and/or laser guidance system and/or apparatus of the present invention are useful for femoral or tibial resection as depicted in FIG. 1.

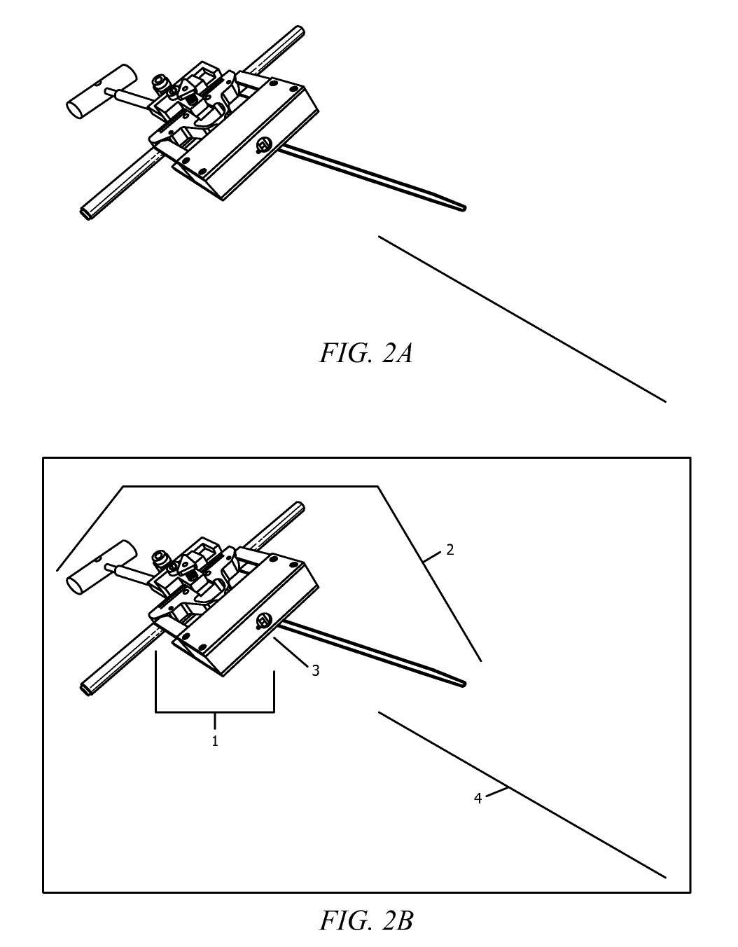

In a further embodiment of the present invention, there is provided a laser guidance system and/or apparatus for femoral resection as depicted in FIG. 2 to FIG. 4. In yet a further embodiment of the present invention, there is provided a laser guidance system and/or apparatus for tibial resection as depicted in FIG. 5 to FIG. 10.

In yet a further embodiment of the present invention, there is provided a surgical method utilizing a laser guidance system and/or apparatus as described herein in bone resection. In another embodiment of the present invention, there is provided a surgical method utilizing a laser guidance system and/or apparatus as described herein in femoral or tibial resection. In a further embodiment, there is provided a surgical method utilizing a laser guidance system and/or apparatus as described herein in femoral or tibial resection comprising the use of a laser guidance system and/or apparatus in accordance with the present invention or as depicted in any of FIG. 2 to FIG. 10.

With regards to the specific Figures, FIG. 1 depicts a schematic showing suggested placement of laser guidance systems on standard cutting blocks for both the femur and tibia. It is within the scope of the present invention that the angle of the laser is adjustable at the cutting block and the angle of the cutting block itself is also adjustable. Further, as shown, it is within the scope of the invention to provide more than one laser on the laser guide, as is shown in the schematic depicting a laser guide attached to a cutting block on the tibia, wherein the laser guide comprises both a coronal laser and a sagittal laser.

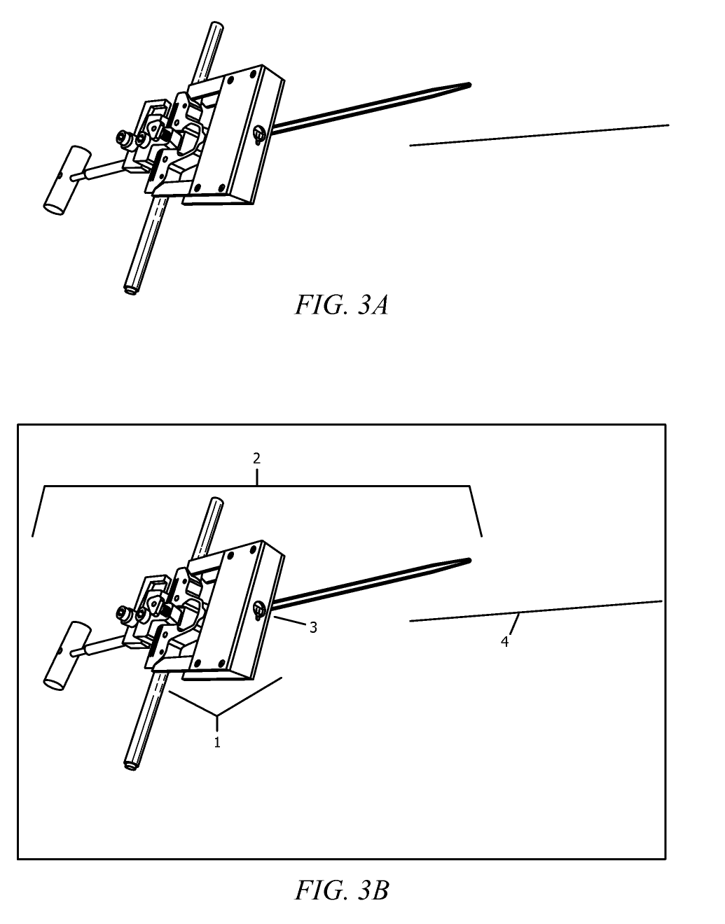

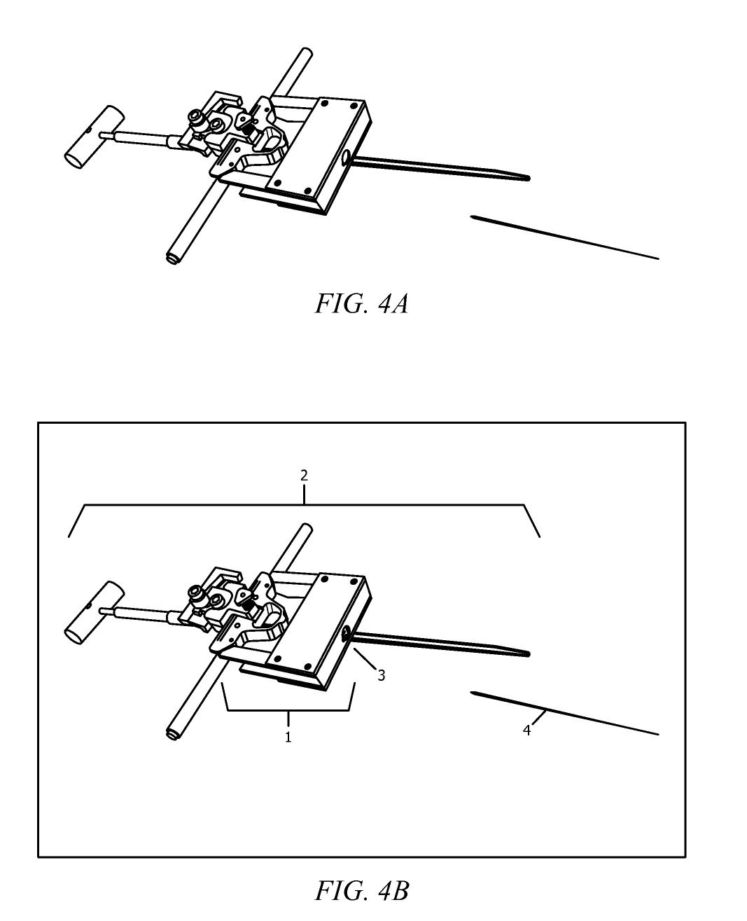

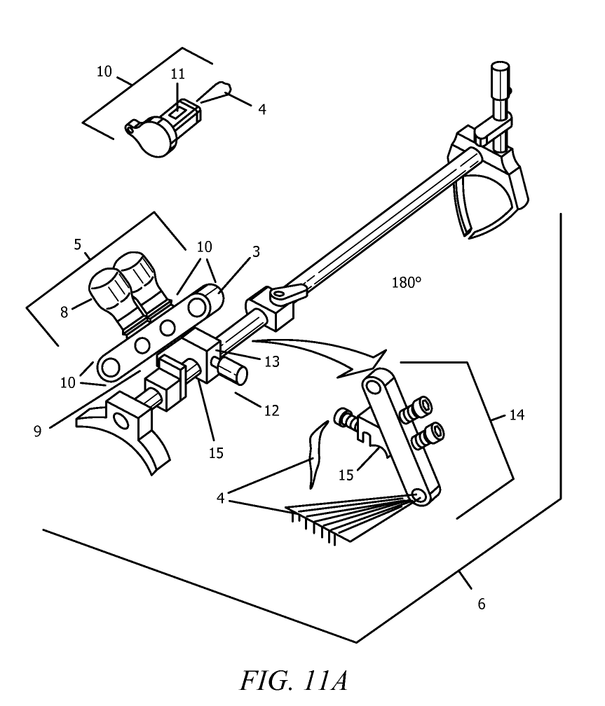

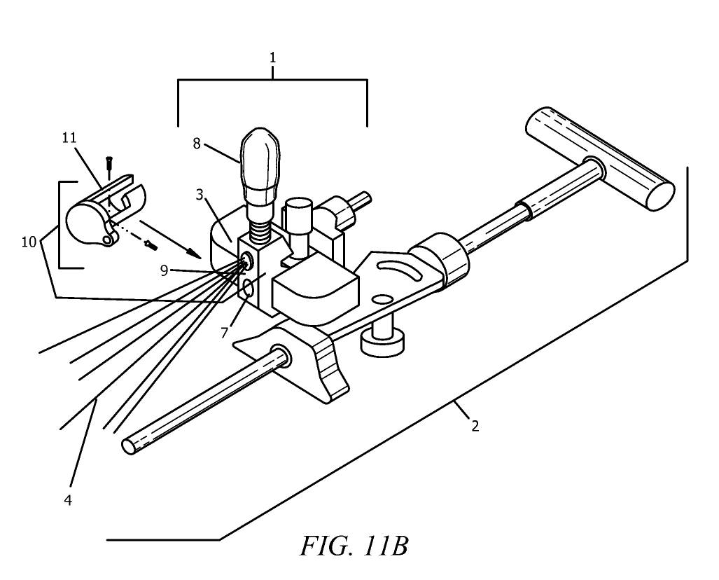

Further embodiments of a femoral laser guide are provided in FIG. 2A, FIG. 2B, FIG. 3A, FIG. 3B, FIG. 4A, and FIG. 4B. Each of these figures provides a different view of a femoral laser guide, wherein there is provided a femoral laser guidance system (1), which is attached to a femoral resection guide (2). The femoral laser guidance system (1) comprises at least one laser (3) which provides at least one laser beam or planar beam (4). A more detailed description of an exemplary femoral laser guide is described below with reference to FIG. 11B. It is noted that the femoral laser guide depicted in FIG. 2A, FIG. 2B, FIG. 3A, FIG. 3B, FIG. 4A, and FIG. 4B is exemplary only. In practice, such a laser guidance system may have a different shape, size or configuration as is suitable for the intended use. For instance, reference is made to the femoral laser guide depicted in FIG. 11B, which while containing similar components, varies in terms of size, shape and configuration.

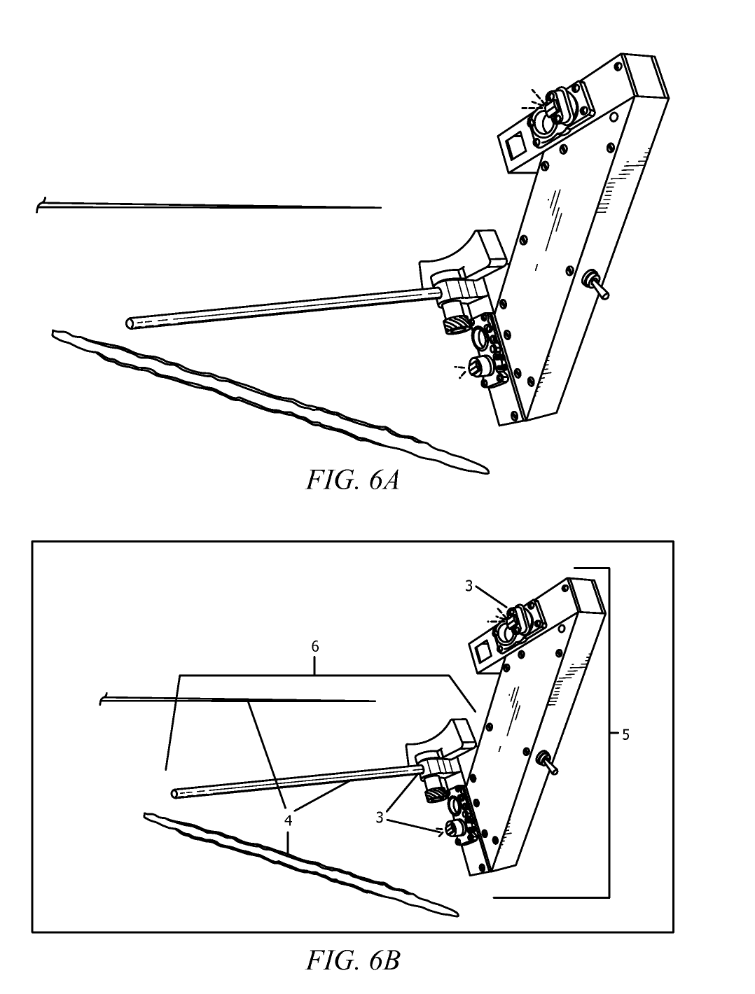

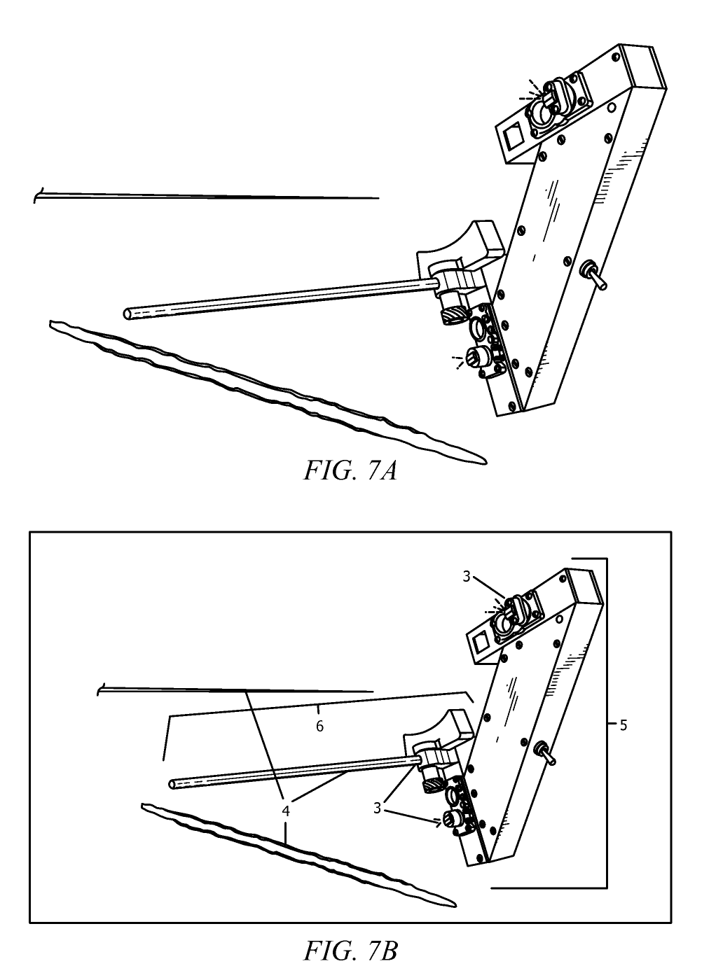

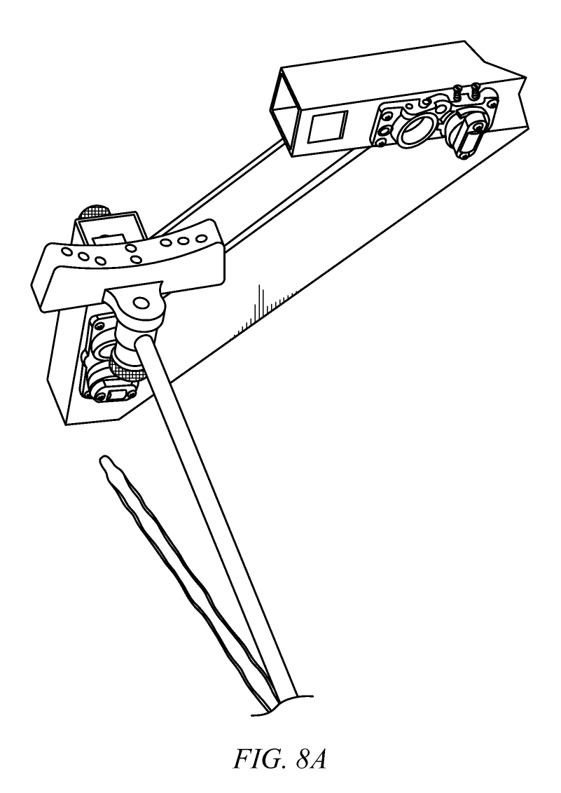

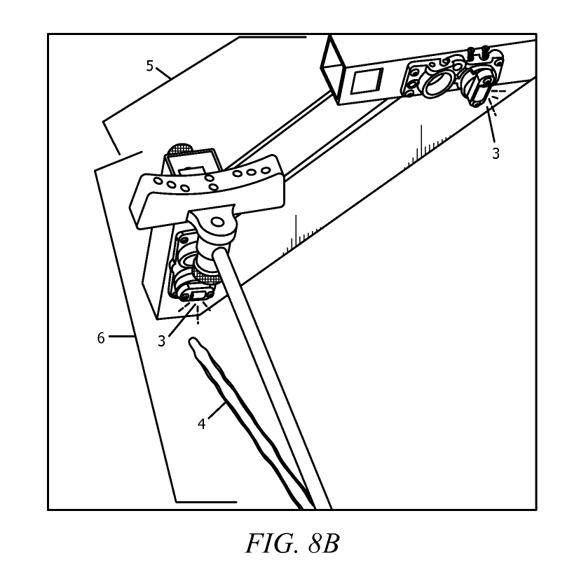

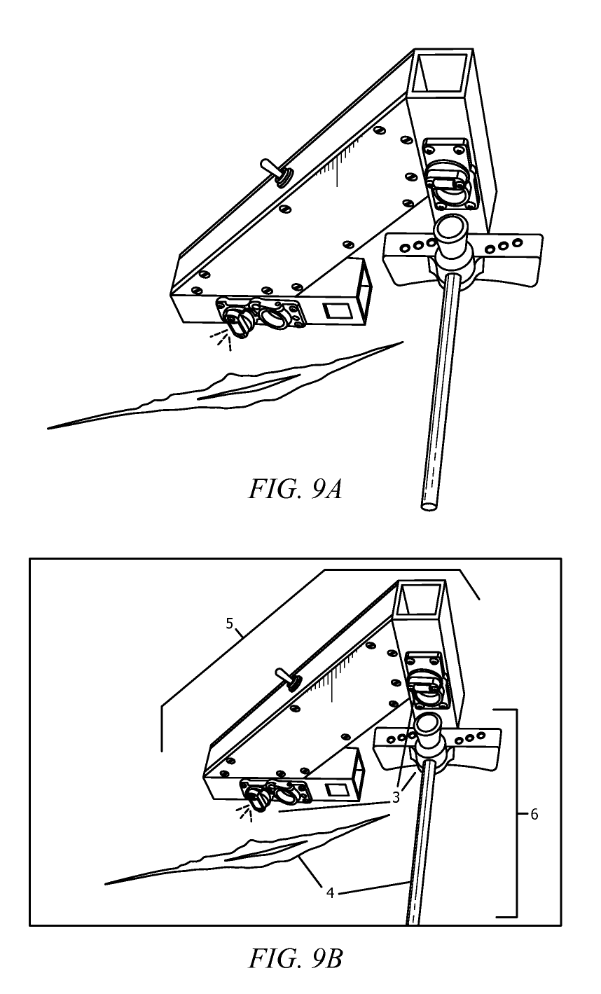

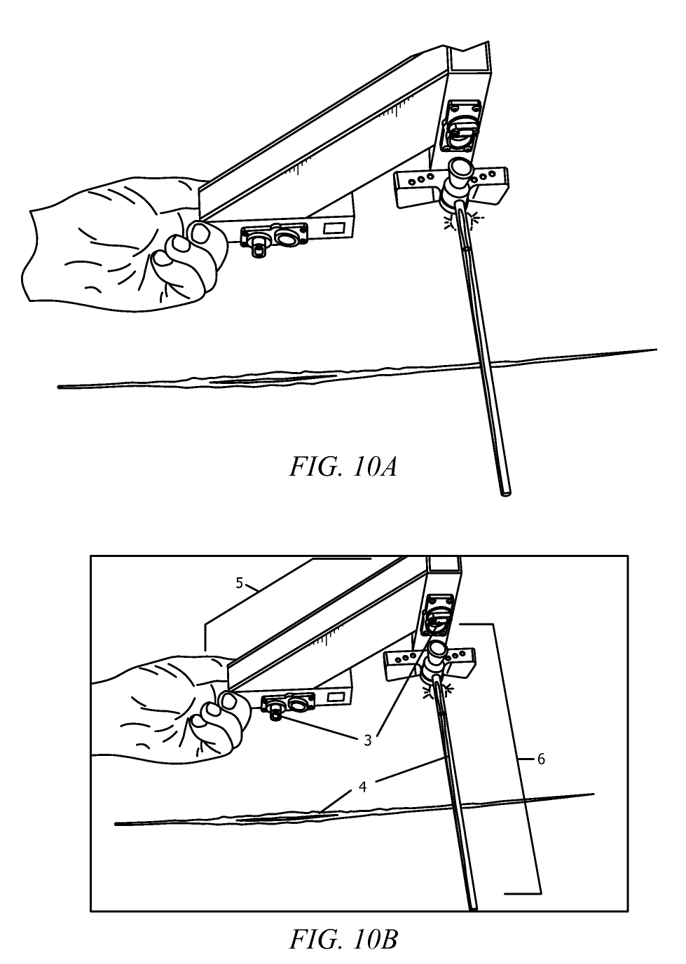

FIG. 5A, FIG. 5B, FIG. 6A, FIG. 6B, FIG. 7A, FIG. 7B, FIG. 8A, FIG. 8B, FIG. 9A, FIG. 9B, FIG. 10A, and FIG. 10B all provide further embodiments of a tibial laser guide from different views. In each of these figures, there is provided a tibial laser guidance system (5), which is attached to a tibial resection guide (6). The tibial laser guidance system (5) comprises at least one laser (3) which provides at least one laser beam or planar beam (4). A more detailed description of an exemplary tibial laser guide is described below with reference to FIG. 11A. It is noted that the tibial laser guide depicted in FIG. 5A, FIG. 5B, FIG. 6A, FIG. 6B, FIG. 7A, FIG. 7B, FIG. 8A, FIG. 8B, FIG. 9A, FIG. 9B, FIG. 10A, and FIG. 10B is exemplary only. In practice, such a laser guidance system may have a different shape, size or configuration as is suitable for the intended use. For instance, reference is made to the tibial laser guide depicted in FIG. 11A, which while containing similar components, varies in terms of size, shape and configuration.

FIG. 11A is a schematic depicting a tibial laser guidance system (5), which is attached to a tibial resection guide (6). The tibial laser guidance system (5) comprises at least one laser (3) which provides at least one laser beam or planar beam (4). The tibial laser guidance system depicted in FIG. 11A comprises a body (13) having removeably attached thereto an arm (14), wherein the arm (14) comprises a laser housing (9). The laser housing (9) further comprises a laser (3) and optionally contains one or more prism mounts (10) containing a prism (11). The one or more prism mounts (10) comprising a prism (11) function to bend, split or provide direction to the laser beam (4) resulting from the laser (3). The one or more prism mounts (10) can be removeable (i.e., via a “poke yoke” or screw tab or other means known in the art) to enable the user to change the prism mount (10) in order to alter or change the laser beam or planar beam provided. The use of more than one prism mounts (10) and/or multiple lasers (3) allows for multiple laser beams or planar beams (4), such that it is possible to utilize more than one beam at a given time, such as a horizontal beam and a vertical beam as depicted in the expanded view of the guidance arm (14) in FIG. 11A.

The laser housing (9) can additionally comprises a power source for the laser (3), such as one or more batteries (8). The arm (14) can be attached to the body (13) by methods known to those of skill in the art, including via a clamp (15). The arm (14) can be rotated or pivoted in a desired amount i.e., from 0-360°, or the arm (14) can be removed from the body (13) and reversed by means of unfastening the attachment means, such as via a clamp (15) that is held in place via a knob (12). Further, the body (13) can be removably attached to the tibial resection guide (6) via means that allow for the body (13) to be moved, including via a clamp (15) that is held in place via a knob (12). Loosening of the clamp (15) by the knob (12) would allow the body (13) to slide along the resection guide (6) as presently depicted. Actual movement of the arm (14) and body (13) can vary depending on the configuration utilized for a given procedure.

FIG. 11B is a schematic depicting a femoral laser guidance system (1), which is attached to a femoral resection guide (2). The femoral laser guidance system (1) comprises at least one laser (3) which provides at least one laser beam or planar beam (4). The femoral laser guidance system depicted in FIG. 11B comprises a laser housing (9). The laser housing (9) further comprises a laser (3) and optionally contains one or more prism mounts (10) containing a prism (11). The one or more prism mounts (10) comprising a prism (11) function to bend, split or provide direction to the laser beam (4) resulting from the laser (3). The one or more prism mounts (10) can be removeable (i.e., via a “poke yoke” or screw tab or other means known in the art) to enable the user to change the prism mount (10) in order to alter or change the laser beam or planar beam provided. The use of more than one prism mounts (10) and/or multiple lasers (3) allows for multiple laser beams or planar beams (4), such that it is possible to utilize more than one beam at a given time.

The laser housing (9) can additionally comprise a power source for the laser (3), such as one or more batteries (8). The femoral laser guidance system (1) can be removably attached to the femoral resection guide (2) via means including a clamp or a magnet (7).

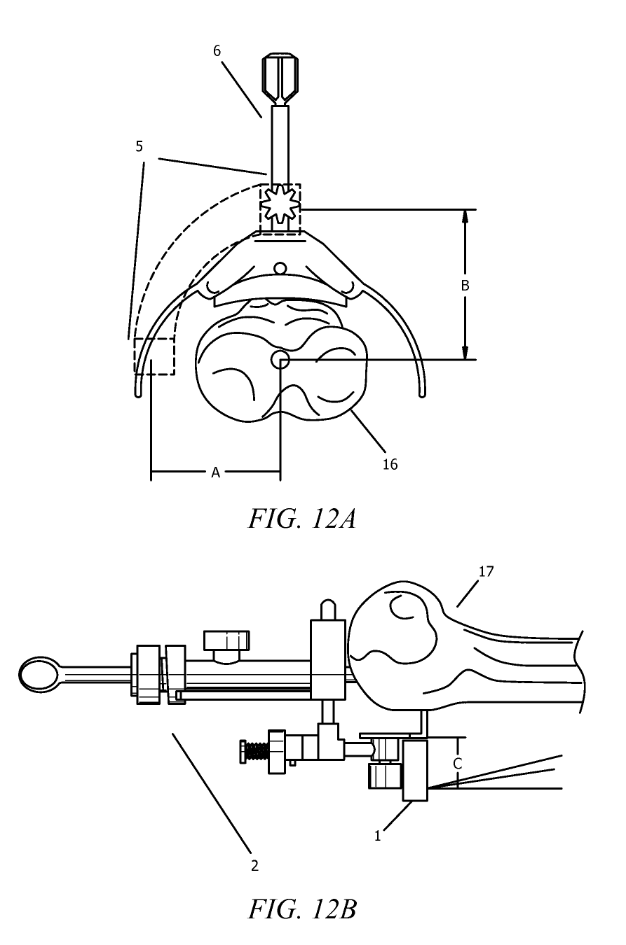

FIG. 12A depicts the placement of a tibial laser guidance system (5) and a tibial resection guide (6) on a tibia (16). The tibial laser guidance system (5) is depicted as the outlined boxes connected by a curved path, which demonstrates the multiple positions the laser (3) can used in for such an application.

FIG. 12B depicts the placement of a femoral laser guidance system (1) and a femoral resection guide (2) on a femur (17). The femoral laser guidance system (1) is depicted as the outlined box, and this demonstrates a potential placement for the laser (3) in relation to the femur (17) as used in such an application.

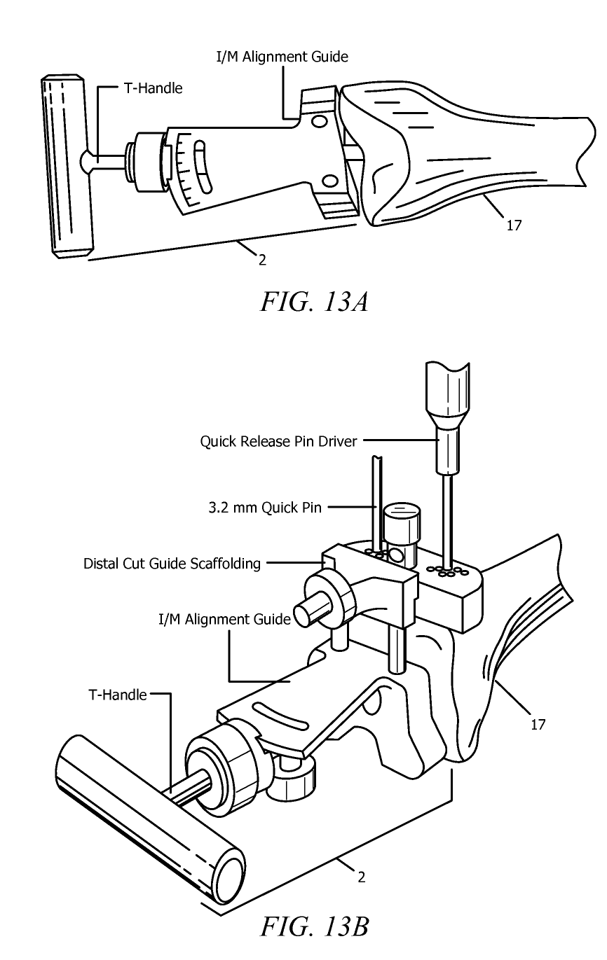

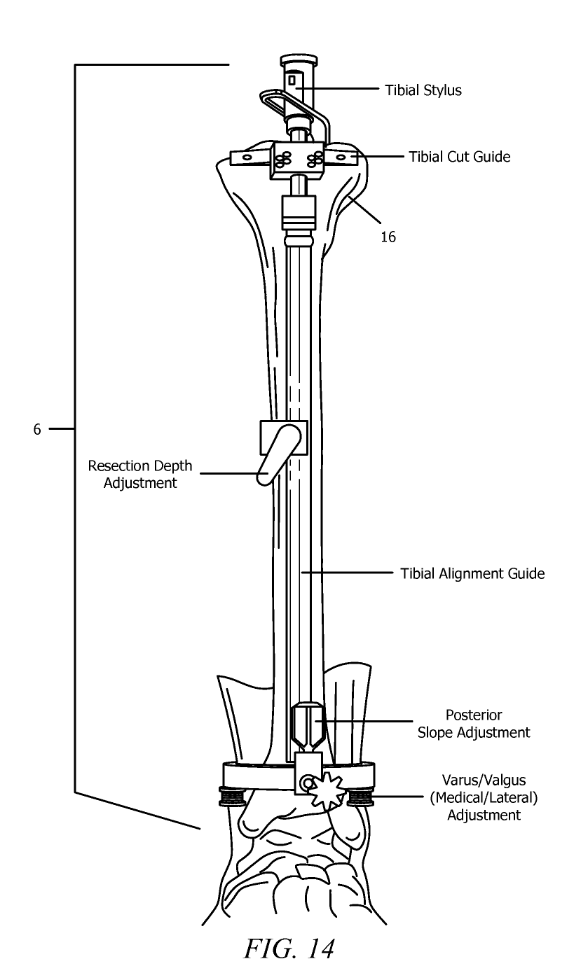

FIGS. 13A and 13B depict two views of a standard femoral resection guide (2) attached to a femur (17). FIG. 14 depicts a views of a standard tibial resection guide (6) attached to a tibia (16). The apparatus of the present invention can be attached to such standard guides as described above, and the present apparatus and methods can be utilized for procedures other than knee arthroplasty where resection of bone or tissue is desired.

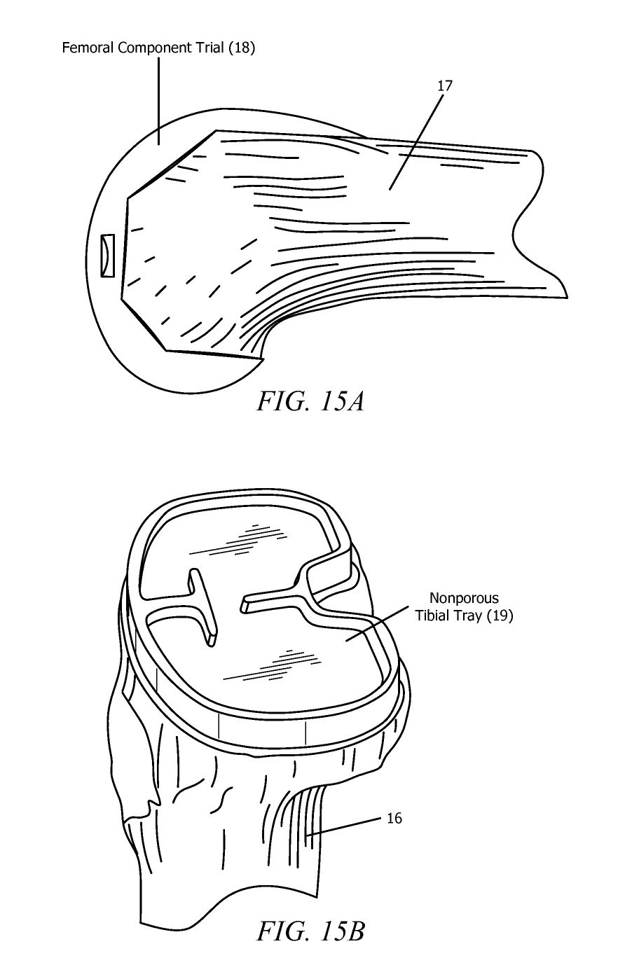

FIGS. 15A and 15B depict a femur (17) and tibia (16), respectively, that have been resected and fit with prosthetic components (18) and (19). The apparatus of the present invention can be utilized to prepare other bones and/or tissue for the attachment of prosthetic devices wherein resection is necessary, including using suitable guides and/or cutting blocks as described above. For instance, the apparatus and methods described herein can be adapted for use in other synovial joints or in other indications as recognized by those of skill in the art.

It is within the scope of the present invention that the laser guidance system and/or apparatus and/or methods provided herein will be useful in obtaining accurate and/or reproducible alignment of replacement joints by providing a means for reproducibly obtaining the required cuts.

It is additionally within the scope of the present invention that the laser guidance system in accordance with the herein description and/or figures can be provided separate from replacement joint kits, or together with a replacement joint kit. To this end, the laser guidance system in accordance with the herein description and/or figures can additionally comprises any number of adaptors or additional parts in order to allow the system to work in conjunction with any available joint replacement kit or resection guides or cutting blocks and/or measuring devices therefore.

Accordingly, it is within the scope of the invention that joint replacement kits and/or resection guides and/or cutting blocks and/or measuring devices can be provided having fewer components for use in the alignment of the replacement joint and/or the resection guide and/or the cutting block and/or measuring device. In one embodiment, the laser guidance system and/or apparatus of the present invention can be used with such joint replacement kits and/or resection guides and/or cutting blocks and/or measuring devices to provide proper alignment such that previous guides and/or systems for alignment and/or instruments used for alignment purposes are unnecessary or the need therefore is reduced.

In a further embodiment, drapes or other appropriate shielding devices or materials are provided which allow the laser guidance system or apparatus of the present invention to be used repeatedly while maintaining or increasing sterility and/or ease of cleaning. Alternatively, the apparatus of the present invention can be manufactured from materials that are compatible with decontamination procedures such as autoclaving, such that the apparatus can be disinfected and re-used. Those of skill in the art would have no difficulty determining materials suitable for such a purpose.

Also within the scope of the invention are kits which may comprise the laser guidance system of the present invention alone, or in combination with resection guides and/or cutting blocks and/or measuring devices and/or joint replacement kits. In a further embodiment, such kids can additionally comprise instructions for use and/or drapes or other protective material or devices for use with the kit.

1. A method of performing a laser assisted total joint arthroplasty comprising the use of a laser guidance system to determine the location and/or angle and/or depth of those cuts in the bone required to fit the replacement joint to a subject in need thereof.

2. The method of claim 1 wherein the laser guidance system comprises one or more lasers, wherein the system or the lasers are directly affixed to a cutting block and/or a measuring device via an attachment means.

3. The method of claim 2 comprising placing the cutting block and/or measuring device over the bone to be cut and aligning the one or more lasers along the desired plane such that the cutting block and/or measuring device is appropriately aligned in relation to the bone.

4. The method of claim 3 wherein the laser is aligned in the sagittal or coronal planes.

5. The method of claim 2, wherein the laser guidance system can be reversed for use on the left or the right side of a patient.

6. The method of claim 5 wherein reversal of the laser guidance system comprises attachment of the laser to the cutting block and/or measuring device with an attaching means having a reverse configuration.

7. The method of claim 2 wherein the laser guidance system additional comprises an attaching means for attaching the laser guidance system to the bone.

8. The method of claim 6 wherein the attaching means is engaged following alignment of the laser to lock the cutting block and/or measuring device into the appropriate position.

9. The method of claim 1 wherein the total joint arthroplasty is selected from one of the following: total knee arthroplasty, total hip arthroplasty, total ankle arthroplasty, and total shoulder arthroplasty.

10. A laser guidance system comprising one or more lasers and an attaching means, wherein the attaching means can be secured to a cutting block and/or a measuring device.

11. The laser guidance system of claim 10, further comprising an additional attaching means whereby the laser guidance system can be secured to the bone.

12. The laser guidance system of claim 10, wherein the system is disposable.

13. An apparatus for guiding the resection of a bone during arthroplasty, comprising:
a) attaching means for anchoring the apparatus to the bone; b) a resection guide or cutting guide operably engaged to said attaching means; and c) means for locating the resection guide relative to the attaching means, said means comprising a laser guide.

14. An apparatus for guiding the resection of a bone during arthroplasty, comprising:
a) a laser guide and b) attaching means for removably attaching said laser guide to a resection guide, wherein the resection guide comprises means for attaching the resection guide to bone.

15. A surgical method in accordance with the description and/or figures provided herein.

16. A laser guidance system in accordance with the description and/or figures provided herein.

17. An apparatus for use in the surgical methods provided herein.
